(19) United States
(12) Patent Application Publication
(10) Pub. No.: US 2024/0008843 A1
(43) Pub. Date: Jan. 11, 2024

(54) PHOTOACOUSTIC CANCER DETECTION AND IMAGING BIOPSY SYSTEM

(71) Applicant: Boston Scientific Scimed, Inc., Maple Grove, MN (US)

(72) Inventor: Meghana Akella, Telangana (IN)

(73) Assignee: Boston Scientific Scimed, Inc., Maple Grove, MN (US)

(21) Appl. No.: 18/348,070

(22) Filed: Jul. 6, 2023

Related U.S. Application Data
(60) Provisional application No. 63/359,075, filed on Jul. 7, 2022.

(51) Int. Cl. A61B 8/12; A61B 10/04; A61B 5/00; A61B 8/08

(52) U.S. Cl. CPC A61B 8/12; A61B 10/04; A61B 5/0095; A61B 8/085; A61B 2010/045

(57) ABSTRACT

The present disclosure describes a medical imaging device with radial ultrasound imaging and light emitting elements arranged for internal imaging of a patient via ultrasound and/or a photo-acoustic effect. The medical imaging device can include both the imaging component and biopsy capability. Further, the medical imaging device can be arranged for insertion into a patient, such as, a lumen of a patent or tissue of a patient.

### CROSS-REFERENCE TO RELATED APPLICATIONS

This application claims the benefit of U.S. Prov. Pat. App. No. 63/359,075, filed Jul. 7, 2022, titled PHOTOACOUSTIC CANCER DETECTION AND IMAGING BIOPSY SYSTEM, which is incorporated herein by reference.

### TECHNICAL FIELD

This disclosure relates generally to the field of medical devices. This disclosure relates to devices, systems and methods that utilize imaging, and more specifically to devices, systems, and methods that integrate imaging, such as ultrasound imaging, and biopsy or other diagnostic and/or therapeutic capability in the same device.

### BACKGROUND

Generally, endoscopic imaging may be performed to determine the internal characteristics of one or more target anatomies. Oftentimes, imaging is used for positioning and/or locating purposes, such as during a diagnostic procedure. For example, an ultrasound imaging device may be inserted into a working channel of the endoscope to image a target anatomy to position a tool through an endoscope for a procedure, such as to biopsy a pulmonary nodule. In such examples, the ultrasound imaging device may be removed from the working channel once the endoscope is positioned and a needle may be inserted into the working channel to biopsy the pulmonary nodule if the endoscope is properly positioned. Challenges with such a procedure may include maintaining location of the nodule when the probe is being exchanged with the needle (e.g., when direct visualization with a bronchoscope may not be locatable at the nodule), controlling orientation of the needle with respect to the nodule, and/or bronchoscope, and having to actuate the biopsy needle into the nodule tissue without the benefit of real-time imaging.

Other diagnostic procedures often rely on external imaging. For example, breast cancer screening often utilized external imaging even where a biopsy is taken. Biopsies are a group of medical diagnostic tests used to determine the structure and composition of tissues or cells. In biopsy procedures, cells or tissues are sampled from an organ or other body part to permit their analysis, for example under microscope. Generally, if an abnormality is found through superficial examination such as palpation or radiographic imaging, a biopsy can be performed to determine the nature of the suspected abnormality.

It is with these considerations in mind that a variety of advantageous medical outcomes may be realized by the devices, systems, and methods of the present disclosure.

### BRIEF SUMMARY

In various embodiments, the present disclosure relates generally to medical imaging devices, such as a real-time visualization. For example, the present disclosure provides a medical tool with radial ultrasound imaging and light emitting elements arranged for internal imaging of a patient via ultrasound and/or a photo-acoustic effect. With some examples, the medical tool can include both photo-acoustic imaging and biopsy capability. For example, the medical tool may include an ergonomic handle and catheter configured for dual-function use during a medical procedure. The medical device may be configured for use with a probe, such as one disposed at the distal end of the catheter and delivered within a working channel of another medical device to provide real-time visualization (e.g., radial ultrasound and photo-acoustic imaging) and manipulation (e.g., diagnostic biopsy sampling) of tissue. As a specific example, one or more components of the medical imaging device may be configured to position a catheter with a first tool/instrument (e.g., radial ultrasound and light emitting element) within a human body (e.g., within a peripheral region of the lung, within a breast of a patient, or the like) to image the tissue (e.g., for cancerous growth, or the like). In addition, the medical device can be configured to position a second tool/instrument (e.g., biopsy needle, or the like) within the catheter to biopsy the imaged tissue.

In some embodiments, a medical device can be provided. The medical device can comprise: a handle assembly comprising a first lumen, a second lumen, and a third lumen; a probe comprising an imaging window, a radiation window, and a port; and a multi-lumen catheter that couples the handle assembly with the probe, wherein the multi-lumen catheter couples to the first lumen to the imaging window, the second lumen to the radiation window, and the third lumen to the port.

With further embodiments, the medical device can include, wherein the first lumen is arranged to receive a radial ultrasound probe such that the radial ultrasound probe can be advanced through the multi-lumen catheter to the imaging window.

With further embodiments, the medical device can include, wherein the second lumen is arranged to receive a fiber optic cable such that the fiber optic cable can be advanced through the multi-lumen catheter to the radiation window.

With further embodiments, the medical device can include, wherein the third lumen is arranged to receive a biopsy needle such that the biopsy needle can be advanced through the multi-lumen catheter to the port.

With further embodiments, the medical device can include, comprising a hub assembly, wherein the fiber optic cable is configured to be optically coupled to a laser source via the hub assembly.

With further embodiments, the medical device can include, wherein the radial ultrasound probe couples to an imaging controller via the hub.

With further embodiments, the medical device can comprise at least one drive cable coupling the radial ultrasound probe to the hub.

With further embodiments, the medical device can comprise a first medical tool, wherein the first medical tool comprises the fiber optic cable and the radial ultrasound probe.

With further embodiments, the medical device can include, wherein the laser source is configured to output laser pulses having a wavelength between 600 nanometers (nm) and 1200 nm.

With further embodiments, the medical device can include, wherein the fiber optic cable is configured to expose a target tissue with the laser pulses via the radiation window.

With further embodiments, the medical device can include, wherein the imaging controller is configured to receive signals indicative of a photo-acoustic effect of the target tissue responsive to exposure to the laser pulses.

With further embodiments, the medical device can include, wherein the handle assembly comprises a flush port.

With further embodiments, the medical device can include, wherein the handle assembly can rotate about the longitudinal axis of the probe.

With further embodiments, the medical device can include, wherein the probe comprises a marker disposed opposite the imaging window.

In some embodiments, the disclosure provides a medical device system that comprises the medical device of the prior described embodiments further including a laser source, an imaging controller, a hub assembly, or combinations thereof.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a medical tool in accordance with at least one embodiment.

FIG. 2 illustrates another medical tool in accordance with at least one embodiment.

FIG. 3 illustrates a portion of a medical tool in accordance with at least one embodiment.

FIG. 4 illustrates a method in accordance with at least one embodiment.

FIG. 5 illustrates another method in accordance with at least one embodiment.

FIG. 6 illustrates a computer-readable storage medium in accordance with at least one embodiment.

FIG. 7 illustrates a computing machine in accordance with at least one embodiment.

### DETAILED DESCRIPTION

Although embodiments of the present disclosure are described with specific reference to assemblies, systems and methods designed to provide dual-function real-time visualization and diagnostic sampling of pulmonary nodules within peripheral regions of the lung, it should be appreciated that such assemblies, systems and methods may be used to visualize and manipulate a variety of tissues within a variety of different body lumens and/or body passages for diagnostic and/or therapeutic purposes. In various embodiments described herein, real-time visualization may refer to imaging with an instrument (e.g., radial ultrasound probe) inserted through a working channel of the endoscope and past the distal end of the endoscope. Additionally, or alternatively, in one or more embodiments described herein, real-time visualization may refer to imaging that does not utilize the visible spectrum of light (e.g., ultrasound imaging or infrared imaging).

The present disclosure is not limited to the embodiments described. The terminology used herein is for the purpose of describing embodiments only and is not intended to be limiting beyond the scope of the appended claims. Unless otherwise defined, all technical terms used herein have the same meaning as commonly understood by one of ordinary skill in the art to which the disclosure belongs.

As used herein, the singular forms “a,” “an,” and “the” are intended to include the plural forms as well, unless the context clearly indicates otherwise. It will be further understood that the terms “comprises” and/or “comprising” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

With general reference to notations and nomenclature used herein, one or more portions of the detailed description which follows may be presented in terms of program procedures executed on a computer or network of computers. These procedural descriptions and representations are used by those skilled in the art to convey the substances of their work most effectively to others skilled in the art. A procedure is here, and generally, conceived to be a self-consistent sequence of operations leading to a desired result. These operations are those requiring physical manipulations of physical quantities. Usually, though not necessarily, these quantities take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in most cases, in any of the operations described herein that form part of one or more embodiments. Rather, these operations are machine operations. Useful machines for performing operations of various embodiments include general purpose digital computers as selectively activated or configured by a computer program stored within that is written in accordance with the teachings herein, and/or include apparatus specially constructed for the required purpose. Various embodiments also relate to apparatus or systems for performing these operations. These apparatuses may be specially constructed for the required purpose or may include a general-purpose computer. The required structure for a variety of these machines will be apparent from the description given.

Reference is now made to the drawings, wherein like reference numerals are used to refer to like elements throughout. In the following description, for purpose of explanation, numerous specific details are set forth to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

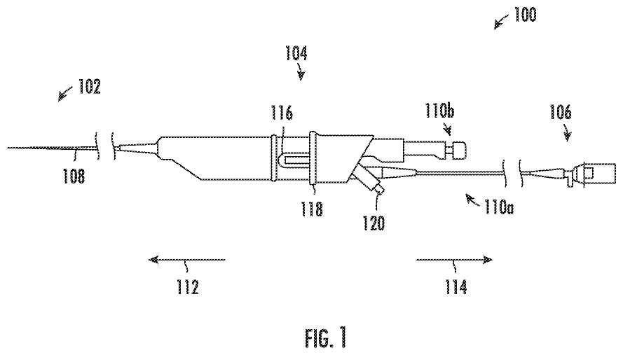

FIG. 1 illustrates a medical imaging device 100 according to one or more embodiments described herein. Generally, medical imaging device 100 may include a probe 102, a handle assembly 104, and a hub assembly 106. The probe 102 may be connected to the handle assembly 104 via a multi-lumen catheter 108, which, among other features, facilitates the efficient and reliable use of a first medical tool 110a and a second medical tool 110b. With some examples, the medical tool 110a may include a radial ultrasound transducer and light emitting element arranged to cause a photo-acoustic effect in tissue and capture images of the tissue responsive to (or while experiencing) the photo-acoustic effect. The medical tool 110b may include a biopsy needle.

The medical imaging device 100 may include a distal end 112 at probe 102 and a proximal end 114 at hub assembly 106. The multi-lumen catheter 108 can include a number (e.g., one or two) lumens in which the medical tool 110a can be disposed and advanced to the distal end 112 of the medical imaging device 100. The multi-lumen catheter 108 can further include another lumen in which the medical tool 110b can be advanced to the distal end 112 of the medical imaging device 100.

The handle assembly 104 may include a tool lock 116, an actuation member 118, and a flush port 120. The actuation member 118 may operate the medical tool 110b between multiple positions when tool lock 116 is unlocked. In one or more embodiments, the hub assembly 106 may interface with logic and/or control circuitry to operate at least the medical tool 110a. For example, medical tool 110a may include one or more light emitters and one or more transducers for imaging, all of which can be interfaced with a controller or control circuitry via hub assembly 106. In many embodiments, one or more components illustrated in FIG. 1, or described with respect thereto, may be the same or similar in construction, function, and/or appearance as one or more other components described herein. Embodiments are not limited in this context.

In various embodiments, the probe 102 may be inserted into a body lumen for diagnostic and/or therapeutic purposes. For example, medical imaging device 100 may be utilized to image and/or biopsy a nodule within a body lumen of a patient. In some embodiments, the medical imaging device 100 may be used as a stand-alone device for insertion into a body lumen. However, in additional, or alternative, embodiments the medical imaging device 100 may be configured to extend through the working channel of another medical device (e.g., a duodenoscope, endoscope, ureteroscope, bronchoscope, colonoscope, arthroscope, cystoscope, hysteroscope, etc.). For instance, medical imaging device 100 may be inserted via a bronchoscope to biopsy lung tissue. In other examples, the multi-lumen catheter 108 can be configured to be inserted into tissue (e.g., breast tissue, or the like).

In many embodiments, the medical imaging device 100 may be modular (include one or more modular assemblies), such as to facilitate efficient manufacturing, selectable tools, and/or reliable operation. In several embodiments, the medical tool 110a and/or medical tool 110b may have a parallel configuration within the handle assembly 104. In several such embodiments, the parallel configuration may facilitate reliable and intuitive single-handed operation with either hand. For example, tool lock 116 may provide ambidextrous operation.

The flush port 120 may facilitate fluid to be provided proximate to the distal end 112, such as via the lumen of the medical tool 110a. In several embodiments, a fluid, such as saline, may be introduced via the flush port 120. In some embodiments, a fluid that assists with imaging may be introduced via the flush port 120, such as a conductive medium that displaces another less conductive medium. For example, saline may be introduced to the distal end of medical imaging device 100 via flush port 120 to enhance the propagation of sound waves from the tissue as compared to air. In some embodiments, the flush port 120 may be used to conduct other types of fluids for various other diagnostic or therapeutic purposes.

The multi-lumen catheter 108 and a proximal portion of the medical tool 110a (e.g., between flush port 120 and hub assembly 106) may have the same, or different, diameters. In some embodiments, a common diameter may be enabled by the fact that the proximal portion of the medical tool 110a has a larger diameter drive cable than the distal portion of the medical tool 110a that extends through the multi-lumen catheter 108.

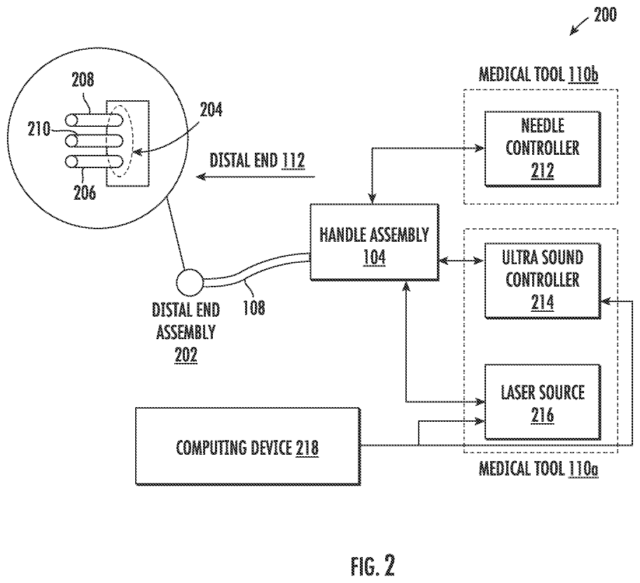

FIG. 2 illustrates a medical imaging device 200 according to one or more embodiments described herein. Medical imaging device 200, like medical imaging device 100, is arranged to be inserted into a working channel of another medical device, such as, an endoscope, bronchoscope, or hollow catheter. Further, medical imaging device 200 can include many of the components of the medical imaging device 100. Medical imaging device 200 includes the handle assembly 104 and the multi-lumen catheter 108. Medical imaging device 200 further includes a distal end assembly 202 coupled to the multi-lumen catheter 108.

The distal end assembly 202 includes several orifices 204 coupled to lumens in the multi-lumen catheter 108. The medical imaging device 200 provides that portions of the medical tool 110a and the medical tool 110b can be introduced into a patient's body via the medical imaging device 200 and multi-lumen catheter 108. Where the medical tool 110a is a photo-acoustic imager, a fiber optic cable 208 and a radial ultrasound transducer 210 can be advanced to the distal end 112 of the medical imaging device 200 via multi-lumen catheter 108 and introduced into the patient's body through orifices 204. As another example, where the medical tool 110b is a biopsy needle, a needle indicator 206 (or biopsy needle) can be advanced to the distal end 112 of the medical imaging device 200 via multi-lumen catheter 108 and introduced into the patient's body through orifice 204.

As contemplated herein, the medical imaging device 200 will include medical tool 110a and medical tool 110b, which will each include respective control assemblies and/or circuitry. For example, the medical tool 110a can include an ultrasound controller 214 and a laser source 216. For example, the laser source 216 can be coupled to a fiber optic cable 208. During operation, the fiber optic cable 208 can be advanced to the distal end 112 of the multi-lumen catheter 108 and the laser source 216 can be activated to irradiate tissue with pulses of laser radiation to cause a photo-acoustic effect in the tissue. Simultaneously, the photo-acoustic effect can be captured by the radial ultrasound transducer 210 and images of the tissue can be generated based on the signals captures by the radial ultrasound transducer 210.

More specifically, as noted, the laser source 216 can be a pulsed laser source. As such, during operation, the tissue can be illuminated or irradiated with pulses of laser light via fiber optic cable 208. The pulses of laser light will cause the tissue to heat and cool. The repeated heating and cooling of the tissue creates a series of expansions and contractions, which may result in an ultrasonic wave that can be detected by the radial ultrasound transducer 210. The ultrasound controller 214 can be coupled to the radial ultrasound transducer 210 and arranged to receive signals from the radial ultrasound transducer 210 indicative of the ultrasonic wave exhibited by the tissue when irradiated by the pulsed laser emitted by the laser source 216 and delivered by the fiber optic cable 208.

Additionally, the medical imaging device 200 can include the medical tool 110b, which can be a biopsy needle. The medical tool 110b can have a needle controller 212 arranged to actuate the biopsy needle to capture a sample of tissue for biopsy. With some examples, the needle controller 212 can be a mechanical actuator and can be integrated into the handle assembly 104.

Importantly, the present disclosure provides both the radial ultrasound transducer 210 and the fiber optic cable 208 insertable into a patient's body to perform internal imaging using the principles of irradiating tissue with pulses of laser light to cause heating and cooling of tissue such that ultrasonic waves resulting from the heating and cooling can be detected. As such, the present disclosure provides a single portable imaging system that can detect changes in tissue and/or cell properties via a catheter.

Laser source 216 can be any of a variety of laser sources and can include diode lasers, gas laser, etc. The laser source 216 can be a pulsed laser source arranged to emit laser light of a variety of wavelengths. For example, the laser source 216 can be arranged to emit pulses of laser having a wavelength between 600 nanometers (nm) and 1200 nm. Ultrasound controller 214 may include logic (circuitry, memory comprising instructions executable by the circuitry, or the like) arranged to control one or more of the calibration, frequency, resolution, translation, interpretation, integration, analysis, and/or display of images captured via the radial ultrasound transducer 210 and the photo-acoustic effect resulting from irradiation of tissue by the light emitted by the fiber optic cable 208 and laser source 216. Further, in some examples, ultrasound controller 214 can include a drive cable arranged to mechanically spin the radial ultrasound transducer 210.

Medical imaging device 200 can further include computing device 218 coupled to one or more of the medical tools (e.g., medical tool 110a, medical tool 110b, etc.). In general, computing device 218 can be any of a variety of computing devices. An example computing device is described later. However, for clarity, computing device 218 may comprise a processor and/or processing circuitry, memory, input and/or output controls, etc. all configured to facilitate interactions with and usage of the medical tools 110a and/or 110b.

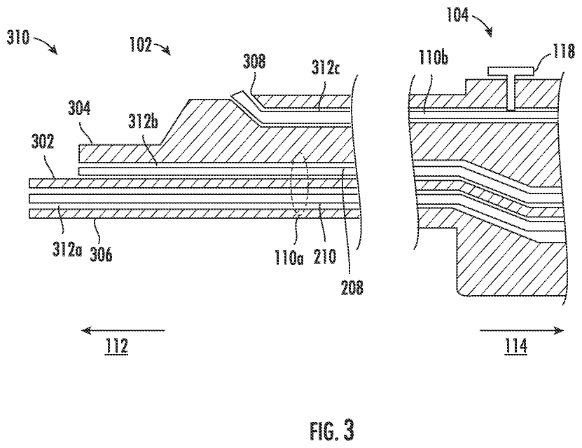

FIG. 3 illustrate an embodiment of medical imaging device 100 comprising multiple lumens as described above. As depicted, the probe 102 may include imaging window 302, radiation window 304, marker 306, side port 308, and the handle assembly 104 may include actuation member 118. The medical imaging device 100 further incudes lumens 312a, 312b, and 312c extending approximately between the distal end 112 of probe 102 and the proximal end 114 of the handle assembly 104. More specifically, a first lumen 312a may include a distal opening in or at the distal end 112 of the probe 102 and a second lumen 312b may include a distal opening in or at the distal end 112 of the probe 102. Further, the medical imaging device 100 includes a third lumen 312c with a distal opening at the distal end 112 of the probe 102. With some examples, one or more of the lumen may be capped or sealed at the distal end. For example, lumen 312a and/or lumen 312b may be capped by a balloon at the distal end 112.

In various embodiments, the first medical tool 110a may be disposed in the first lumen 312a and the second lumen 312b while the second medical tool 110b may be disposed in the third lumen 312c. As described above, the medical tool 110a may include a photo-acoustic imagers including radial ultrasound transducer 210 and fiber optic cable 208 while the medical tool 110b may include a biopsy needle.

In the illustrated embodiment, probe 102 includes the radiation window 304. In general, radiation window 304 can be any area of probe 102 that is transmissive to the wavelengths of light emitted by the laser source 216 and ultimately emitted by the distal end of the fiber optic cable 208. With some examples, the radiation window 304 can be an open area of the distal end of probe 102 while in other areas the radiation window 304 can be a material having a high transmission coefficient, such as, for example, glass, clear plastic, or the like.

Additionally, the probe 102 includes imaging window 302 and marker 306. In many embodiments, imaging window 302 may refer to one or more portions of the probe 102 that are substantially transparent to the imaging energy wave lengths while marker 306 may refer to one or more portions of the probe that are relatively opaque to the imaging energy wave lengths. Marker 306 may comprise any medium that absorbs imaging energy wavelengths (e.g., ultrasound waves). For example, metal or metal alloys (e.g., stainless steel or nitinol) may be used. In some embodiments, non-metals may be used, such as air pockets embedded in the wall of the imaging window. In various embodiments, the marker 306 may be radiopaque, such as to show up on x-ray and/or fluoroscopic imaging.

In such embodiments, marker 306 may be positioned to indicate in a generated image where the second medical tool 110b would be positioned when actuation member 118 is moved to cause axial displacement in medical tool 110b, resulting in medical tool 110b extending out of side port 308. To position the probe 102 based on generated images, the handle assembly 104 may be rotated along an axial rotation to cause probe 102 to rotate. For example, the handle assembly 104 may be rotated to align the side port 308 with a tissue target nodule based on indications of marker 306 in generated images. In some such examples, once aligned, actuation member 118 may be moved distally to cause the distal end of the medical tool 110b to contact and/or penetrate the target tissue. In various embodiments, a marker 306 may be embedded in a wall of a lumen, such as the wall of the lumen 312a. As will be appreciated, device rotation (e.g., orientation of the marker and the needle radially) may enable more efficient biopsying of eccentric tissue (e.g., when biopsying target tissue that has irregular margins, is of an asymmetric shape, does not extend around an entire circumference of the body lumen, and the like) where control or orientation and position of the needle may be more critical.

As an example, marker 306 may be oriented around the circumference of the imaging window at a known angle from side port 308. In such a case (e.g., when targeting a lung nodule for core biopsy) marker 306 may be oriented on the radial ultrasound image at the known angle from the intended biopsy site, so that a needle exiting side port 308 will be correctly aligned with the biopsy site. In a further such example, the marker 306 may be oriented on the radial ultrasound image 180 degrees across from the intended biopsy site. In many embodiments, the known angle from the intended biopsy site may be configured such that tolerances may be provided. For example, the marker 306 may be oriented on the radial ultrasound image 180±35 degrees from the intended biopsy site.

Likewise, the radiation window 304 may be oriented radially in-line with the intended biopsy site. For example, the radiation window 304 may be oriented on the radial ultrasound image 0 degrees across from the intended biopsy site, ±35 degrees from the intended biopsy site, or the like. As such, tissue intended for biopsy can be phot-acoustically imaged as described herein.

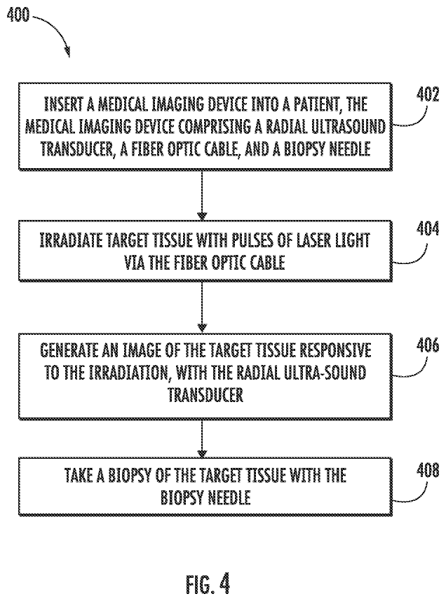

FIG. 4 illustrates a method 400 in accordance with various embodiments of the present disclosure for imaging tissue and/or biopsying tissue. The method 400 is described with reference to the medical imaging device 100 described above. It is to be appreciated however, that the method 400 could be implemented using a different medical imaging device than that described herein. Embodiments are not limited in this context. Further, method 400 could be implemented with different combinations of operations than that depicted in FIG. 4. For example, a method like method 400 could be implemented to include additional blocks, an alternative arrangement of blocks, or omit some blocks from the method 400 depicted and described herein.

The method 400 can begin at block 402, “insert a medical imaging device into a patient, the medical imaging device comprising a radial ultrasound transducer, a fiber optic cable, and a biopsy needle,” wherein a physician can insert into a patient (e.g., via another medical device, directly, or the like) probe 102 including medical tool 110a and medical tool 110b. Where, the probe 102 is inserted into the patient through another medical tool (e.g., a bronchoscope, or the like), the physician can, at block 402, extend the probe 102 past the end of the other medical tool.

Continuing to block 404, “irradiate target tissue with pulses of laser light via the fiber optic cable,” the target tissue can be irradiated with pulses of laser light (e.g., from the laser source 216) via the fiber optic cable 208. As outlined above, the pulses of laser light irradiating the target tissue cause the target tissue to expand and contract and to exhibit photo-acoustic effects, or rather, to emit photo-acoustic vibrations based on the expansion and contraction. For example, one application is the detection of cancerous cells or cancerous growths. Due to the increased blood flow at cancerous sites, the cancerous target tissue will exhibit a different photo-acoustic effect than non-cancerous tissue. This difference in effect may be visible via photo-acoustic imaging.

To that end, method 400 can continue to block 406, “generate an image of the target tissue responsive to the irradiation, with the radial ultrasound transducer,” where images can be generated from the radial ultrasound transducer 210 responsive to the irradiation of the tissue by the laser source 216 and the fiber optic cable 208 (e.g., at block 404). In some embodiments, blocks block 404 and block 406 can be performed simultaneously.

The method 400 may optionally include block 408, “take a biopsy of the target tissue with the biopsy needle,” wherein a biopsy of the target tissue can be taken with the medical tool 110b.

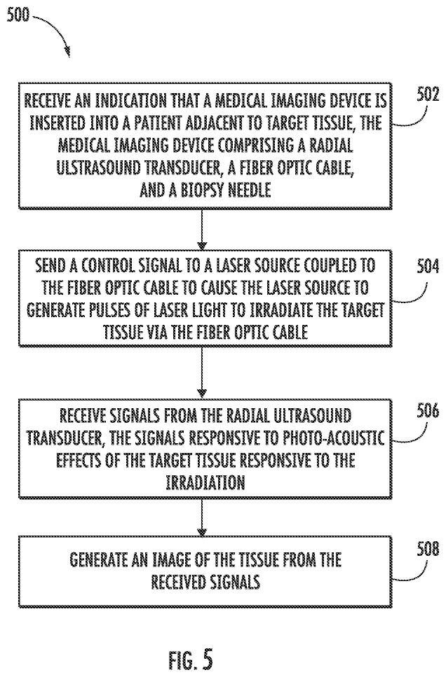

FIG. 5 illustrates a method 500 that can be implemented by a medical device according to various examples of the present disclosure. The method 500 is described with reference to the medical imaging device 100 described above. It is to be appreciated however, that the method 500 could be implemented using a different medical imaging device than that described herein. Embodiments are not limited in this context. Further, method 500 could be implemented with different combinations of operations than that depicted in FIG. 5. For example, a method like method 500 could be implemented to include additional blocks, an alternative arrangement of blocks, or omit some blocks from the method 500 depicted and described herein.

The method 500 can begin at block 502, “receive an indication that a medical imaging device is inserted into a patient adjacent to target tissue, the medical imaging device comprising a radial ultrasound transducer, a fiber optic cable, and a biopsy needle,” where laser source 216 and/or ultrasound controller 214 can receive an indication that the probe 102 including medical tool 110a and optionally, medical tool 110b, is placed adjacent to a target tissue (e.g., in a lung, in breast tissue, or the like). For example, laser source 216 and/or ultrasound controller 214 can receive the indication via an input button actuated by a physician, or the like. As a specific example, computing device 218 can receive an indication (from an input button, from a user interface selection, from a touch screen, or the like) where the indication indicates that the medical imaging device is inserted into the patient.

Continuing to block 504, “send a control signal to a laser source coupled to the fiber optic cable to cause the laser source to generate pulses of laser light to irradiate the target tissue via the fiber optic cable,” and to block 506, “receive signals from the radial ultrasound transducer, the signals responsive to photo-acoustic effects of the target tissue responsive to the irradiation;” at block 504 and block 506, computing device 218 can send a control signal to laser source 216 where the control signal causes the laser source 216 to activate and generate pulses of laser energy to be delivered to target tissue via the fiber optic cable 208.

Similarly, the computing device 218 can send and/or receive signals from the ultrasound controller 214 causing the ultrasound controller 214 to activate the radial ultrasound transducers 210 and capture indications of photo-acoustic effects manifest by the target tissue resulting from exposure to the pulses of laser light.

Continuing to block 508, “generating an image of the tissue from the received signals,” the computing device 218 can generate ultrasound and/or photo-acoustic images from the signals received from the ultrasound controller 214.

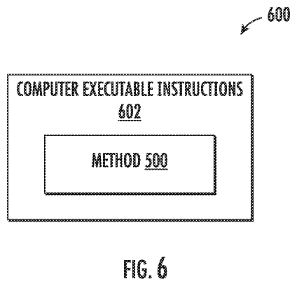

FIG. 6 illustrates computer-readable storage medium 600. Computer-readable storage medium 600 may comprise any non-transitory computer-readable storage medium or machine-readable storage medium, such as an optical, magnetic or semiconductor storage medium. In various embodiments, computer-readable storage medium 600 may comprise an article of manufacture. In some embodiments, computer-readable storage medium 600 may store computer executable instructions 602 with which circuitry (e.g., laser source 216, ultrasound controller 214, or the like) can execute. For example, computer executable instructions 602 can include instructions to implement operations described with respect to method 500. Examples of computer-readable storage medium 600 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 602 may include any suitable type of code, such as source code, compiled code, interpreted code, executable code, static code, dynamic code, object-oriented code, visual code, and the like.

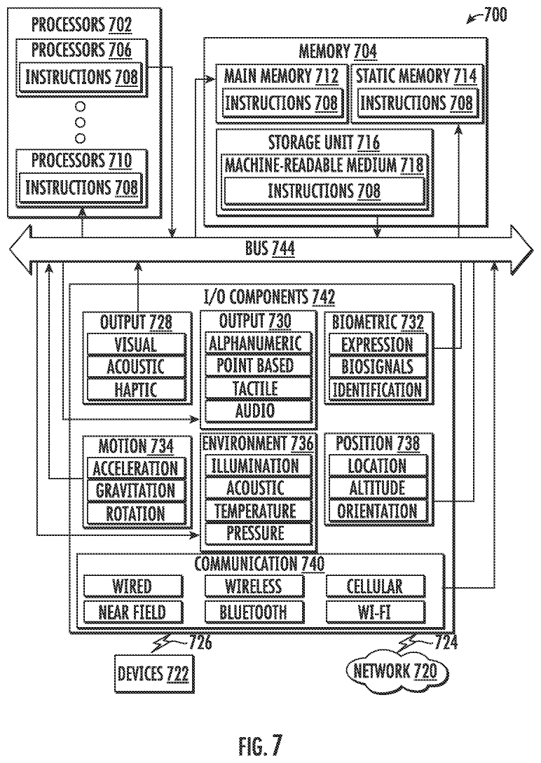

FIG. 7 illustrates a diagrammatic representation of a machine 700 in the form of a computer system within which a set of instructions may be executed for causing the machine to perform any one or more of the methodologies discussed herein. More specifically, FIG. 7 shows a diagrammatic representation of the machine 700 in the example form of a computer system, within which instructions 708 (e.g., software, a program, an application, an applet, an app, or other executable code) for causing the machine 700 to perform any one or more of the methodologies discussed herein may be executed. For example, the instructions 708 may cause the machine 700 to execute method 500 of FIG. 5, or the like. More generally, the instructions 708 may cause the machine 700 to activate a medical tool (e.g., medical tool 110b, ultrasound controller 214 and/or laser source 216) and to generate ultrasound and/or photo-acoustic images.

The instructions 708 transform the general, non-programmed machine 700 into a particular machine 700 programmed to carry out the described and illustrated functions in a specific manner. In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 708, sequentially or otherwise, that specify actions to be taken by the machine 700. Further, while only a single machine 700 is illustrated, the term “machine” shall also be taken to include a collection of machines 200 that individually or jointly execute the instructions 708 to perform any one or more of the methodologies discussed herein.

The machine 700 may include processors 702, memory 704, and I/O components 742, which may be configured to communicate with each other such as via a bus 744. In an example embodiment, the processors 702 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 706 and a processor 710 that may execute the instructions 708. The term “processor” is intended to include multi-core processors that may comprise two or more independent processors (sometimes referred to as “cores”) that may execute instructions contemporaneously. Although FIG. 7 shows multiple processors 702, the machine 700 may include a single processor with a single core, a single processor with multiple cores (e.g., a multi-core processor), multiple processors with a single core, multiple processors with multiples cores, or any combination thereof.

The memory 704 may include a main memory 712, a static memory 714, and a storage unit 716, both accessible to the processors 702 such as via the bus 744. The main memory 704, the static memory 714, and storage unit 716 store the instructions 708 embodying any one or more of the methodologies or functions described herein. The instructions 708 may also reside, completely or partially, within the main memory 712, within the static memory 714, within machine-readable medium 718 within the storage unit 716, within at least one of the processors 702 (e.g., within the processor's cache memory), or any suitable combination thereof, during execution thereof by the machine 700.

The I/O components 742 may include a wide variety of components to receive input, provide output, produce output, transmit information, exchange information, capture measurements, and so on. The specific I/O components 742 that are included in a particular machine will depend on the type of machine. For example, portable machines such as mobile phones will likely include a touch input device or other such input mechanisms, while a headless server machine will likely not include such a touch input device. It will be appreciated that the I/O components 742 may include many other components that are not shown in FIG. 7. The I/O components 742 are grouped according to functionality merely for simplifying the following discussion and the grouping is in no way limiting. In various example embodiments, the I/O components 742 may include output components 728 and input components 730. The output components 728 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 730 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 742 may include biometric components 732, motion components 734, environmental components 736, or position components 738, among a wide array of other components. For example, the biometric components 732 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 734 may include acceleration sensor components (e.g., accelerometer), gravitation sensor components, rotation sensor components (e.g., gyroscope), and so forth. The environmental components 736 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 738 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 742 may include communication components 740 operable to couple the machine 700 to a network 720 or devices 722 via a coupling 724 and a coupling 726, respectively. For example, the communication components 740 may include a network interface component or another suitable device to interface with the network 720. In further examples, the communication components 740 may include wired communication components, wireless communication components, cellular communication components, Near Field Communication (NFC) components, Bluetooth ° components (e.g., Bluetooth ° Low Energy), WiFi® components, and other communication components to provide communication via other modalities. The devices 722 may be another machine or any of a wide variety of peripheral devices (e.g., a peripheral device coupled via a USB).

Moreover, the communication components 740 may detect identifiers or include components operable to detect identifiers. For example, the communication components 740 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 740, such as location via Internet Protocol (IP) geolocation, location via Wi-Fi® signal triangulation, location via detecting an NFC beacon signal that may indicate a particular location, and so forth.

The various memories (i.e., memory 704, main memory 712, static memory 714, and/or memory of the processors 702) and/or storage unit 716 may store one or more sets of instructions and data structures (e.g., software) embodying or utilized by any one or more of the methodologies or functions described herein. These instructions (e.g., the instructions 708), when executed by processors 702, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 720 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 720 or a portion of the network 720 may include a wireless or cellular network, and the coupling 724 may be a Code Division Multiple Access (CDMA) connection, a Global System for Mobile communications (GSM) connection, or another type of cellular or wireless coupling. In this example, the coupling 724 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 708 may be transmitted or received over the network 720 using a transmission medium via a network interface device (e.g., a network interface component included in the communication components 740) and utilizing any one of several well-known transfer protocols (e.g., hypertext transfer protocol (HTTP)). Similarly, the instructions 708 may be transmitted or received using a transmission medium via the coupling 726 (e.g., a peer-to-peer coupling) to the devices 722. The terms “transmission medium” and “signal medium” mean the same thing and may be used interchangeably in this disclosure. The terms “transmission medium” and “signal medium” shall be taken to include any intangible medium that can store, encoding, or carrying the instructions 708 for execution by the machine 700, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A medical device, comprising:
a handle assembly comprising a first lumen, a second lumen, and a third lumen;
a probe comprising an imaging window, a radiation window, and a port; and
a multi-lumen catheter that couples the handle assembly with the probe,
wherein the multi-lumen catheter couples to the first lumen to the imaging window, the second lumen to the radiation window, and the third lumen to the port.

2. The medical device of claim 1, wherein the first lumen is arranged to receive a radial ultrasound probe such that the radial ultrasound probe can be advanced through the multi-lumen catheter to the imaging window.

3. The medical device of claim 2, wherein the second lumen is arranged to receive a fiber optic cable such that the fiber optic cable can be advanced through the multi-lumen catheter to the radiation window.

4. The medical device of claim 3, wherein the third lumen is arranged to receive a biopsy needle such that the biopsy needle can be advanced through the multi-lumen catheter to the port.

5. The medical device of claim 4, comprising a hub assembly, wherein the fiber optic cable is configured to be optically coupled to a laser source via the hub assembly.

6. The medical device of claim 5, wherein the radial ultrasound probe couples to an imaging controller via the hub.

7. The medical device of claim 6, comprising at least one drive cable coupling the radial ultrasound probe to the hub.

8. The medical device of claim 7, comprising a first medical tool, wherein the first medical tool comprises the fiber optic cable and the radial ultrasound probe.

9. The medical device of claim 8, wherein the laser source is configured to output laser pulses having a wavelength between 600 nanometers (nm) and 1200 nm.

10. The medical device of claim 9, wherein the fiber optic cable is configured to expose a target tissue with the laser pulses via the radiation window.

11. The medical device of claim 10, wherein the imaging controller is configured to receive signals indicative of a photo-acoustic effect of the target tissue responsive to exposure to the laser pulses.

12. The medical device of claim 1, the handle assembly comprising a flush port.

13. The medical device of claim 1, wherein the handle assembly can rotate about the longitudinal axis of the probe.

14. The medical device of claim 1, the probe comprising a marker disposed opposite the imaging window.

15. A medical system, comprising:
a handle assembly, comprising:
a first lumen, a second lumen, and a third lumen;
a probe comprising an imaging window, a radiation window, and a port; and
a multi-lumen catheter that couples the handle assembly with the probe,
wherein the multi-lumen catheter couples the first lumen to the imaging window, the second lumen to the radiation window, and the third lumen to the port;
a hub assembly;
a laser source, wherein a fiber optic cable, arranged to be introduced into the second lumen, is configured to be optically coupled to the laser source via the hub assembly; and
an imaging controller, wherein a radial ultrasound probe, arranged to be introduced into the first lumen and advanced through the multi-lumen catheter to the imaging window, is configured to be coupled to the imaging controller via the hub assembly.

16. The medical system of claim 15, wherein the third lumen is arranged to receive a biopsy needle such that the biopsy needle can be advanced through the multi-lumen catheter to the port.

17. The medical system of claim 16, wherein the laser source is configured to output laser pulses having a wavelength between 600 nanometers (nm) and 1200 nm.

18. The medical system of claim 17, wherein the fiber optic cable is configured to expose a target tissue with the laser pulses via the radiation window.

19. The medical system of claim 18, wherein the imaging controller is configured to receive signals indicative of a photo-acoustic effect of the target tissue responsive to exposure to the laser pulses.

20. The medical system of claim 15, the probe comprising a marker disposed opposite the imaging window.
